(19) United States
(12) Patent Application Publication
(10) Pub. No.: US 2013/0047979 A1
(43) Pub. Date: Feb. 28, 2013

(54) METHODS FOR THE RECOVERY OF HCL AND FOR THE PRODUCTION OF CARBOHYDRATES

(71) Applicants: Aharon Eyal, Jerusalem (IL); Robert Jansen, Portela (PT)

(72) Inventors: Aharon Eyal, Jerusalem (IL); Robert Jansen, Portela (PT)

(73) Assignee: HCL CLEANTECH LTD., Tel-Aviv (IL)

(21) Appl. No.: 13/499,638

(22) PCT Filed: Sep. 21, 2010
(86) PCT No.: PCT/IL10/00786

§ 371 Date: Jul. 18, 2012

(30) Foreign Application Priority Data
Oct. 1, 2009 (IL) 201,330
Sep. 19, 2010 (IL) 208,219

(51) Int. Cl. C13K 1/02; C01B 7/07; C09K 3/00

(52) U.S. Cl. 127/37; 252/364; 423/488

(57) ABSTRACT

The invention provides an organic phase composition comprising: a. a first component selected from the group consisting of quaternary amines; b. a second component selected from: b1. The group consisting of category B organic acids; b2. The group consisting of a mixtures of category B organic acids and category C organic acids at a B/C molar ratio of RB/C; and b3. The group consisting of a mixtures of category A organic acids and category C organic acids at an A/C molar ratio of RA/C; c. a third component selected from the group consisting of solvents for said first component and for said second component, wherein (i) all three components are oil-soluble and water-insoluble; (ii) the molar concentration of each of said first component and said second component is greater than 0.6 mol/Kg; (iii) the molar ratio between said second component and said first component is greater than 0.9; (iv) RB/C and RA/c are greater than 2; (v) category A organic acids are selected from the group consisting of poly-aromatic sulfonic acids, naphthalene sulfonic acids and acids with a pKa in the range within +/−0.5 pKa units of that of naphthalene sulfonic acid; (vi) category B organic acids are selected from the group consisting of mono-aromatic sulfonic acids, benzene sulfonic acids, and acids with a pKa in the range within +/−0.5 pKa units of that of benzene sulfonic acid; and (vii) category C organic acids are selected from the group consisting of phosphoric acid esters and acids with a pKa in the range within +/−0.5 pKa units of that of di-octyl esters of phosphoric acid.

The present invention relates to methods for the recovery of HCl and for the production of carbohydrates and to organic phases utilizable therein. More particularly, the present invention relates to production of carbohydrates via hydrolysis of polysaccharides, preferably ones present in lignocellulosic material, using as a catalyst, a highly concentrated hydrochloric acid, e.g. of concentration greater than 39% wt, preferably greater than 40% and more preferably greater than 41% wt.

Preferably other products are also formed, e.g. lignin of low chloride content for burning for energy and for other applications and tall oils.

The present invention also relates to recovering the hydrolyzing acid from the hydrolyzate at minimum loss and at as high a concentration as possible to avoid or minimize the need to re-concentrate hydrochloric acid solutions, particularly to concentrate azeotropic hydrochloric solutions to above-azeotropic concentrations.

The recovery of the acid should be highly selective, so that there is no loss or there is a minimal loss of products on the one hand and no build up of impurities on the other. Another objective is to use solvent extraction in the recovery process and conduct it at a temperature low enough to avoid degradation of the products and of the extractant used in the extraction process.

Other objectives are to minimize the number of solvents used in the process and to minimize production costs.

According to a first aspect of the present invention there is provided an organic phase composition comprising:
a. a first component selected from the group consisting of quaternary amines
b. a second component selected from
   b1. the group consisting of category B organic acids;
   b2. the group consisting of a mixtures of category B organic acids and category C organic acids at a B/C molar ratio of R_{B/C};
   b3. the group consisting of a mixtures of category A organic acids and category C organic acids at an A/C molar ratio of R_{A/C};
c. a third component selected from the group consisting of solvents for said first component and for said second component, wherein
   (i) all three components are oil-soluble and water-insoluble;
   (ii) the molar concentration of each of said first component and said second component is greater than 0.6 mol/Kg;
   (iii) the molar ratio between said second component and said first component is greater than 0.9;
   (iv) R_{B/C} and R_{A/C} are greater than 2;
   (v) category A organic acids are selected from the group consisting of poly-aromatic sulfonic acids, naphthalene sulfonic acids and acids with a pKa in the range within +/−0.5 pKa units of that of naphthalene sulfonic acid;
   (vi) category B organic acids are selected from the group consisting of mono-aromatic sulfonic acids, benzene sulfonic acids, and acids with a pKa in the range within +/−0.5 pKa units of that of benzene sulfonic acid; and
   (vii) category C organic acids are selected from the group consisting of phosphoric acid esters and acids with a pKa in the range within +/−0.5 pKa units of that of di-octyl esters of phosphoric acid.

In preferred embodiments of said aspect of the invention, the organic phase composition, further comprises HCl wherein the molar ratio between HCl and said first component is greater than 0.2.

In preferred embodiments of said aspect of the invention, the organic phase composition, further comprises HCl wherein the molar ratio between HCl and said first component is greater than 1.0.

Preferably said third component is composed of at least 70% wt. hydrocarbon and said hydrocarbon is an aliphatic hydrocarbon, an aromatic hydrocarbon or a combination thereof.

Said organic phase composition is preferably further characterized in that when equilibrated at 45° C. with an aqueous solution containing 35% wt. dextrose and 1% wt. HCl, said organic phase is loaded to at least 0.05 mol/Kg, preferably to at least 0.10 mol/Kg, more preferably to at least 0.15 mol/Kg, and most preferably to at least 0.20 mol/Kg, and in that when equilibrated at 90° C. with an aqueous solution containing 2% wt. HCl, said organic phase is loaded to less than 0.30 mol/Kg, preferably to less than 0.25 mol/Kg, more preferably to less than 0.20 mol/Kg and most preferably to less than 0.15 mol/Kg.

Said organic phase composition is preferably further characterized in that when equilibrated at 45° C. with an aqueous solution containing 35% wt. dextrose and 5% wt. HCl, said organic phase is loaded to between 0.10 mol/Kg and 0.50 mol/Kg, preferably between 0.15 mol/Kg and 0.45 mol/Kg, more preferably between 0.20 mol/Kg and 0.40 mol/Kg, in that when equilibrated at 45° C. with an aqueous solution containing 35% wt. dextrose and 10% wt. HCl, said organic phase is loaded to between 0.20 mol/Kg and 0.70 mol/Kg, preferably between 0.25 mol/Kg and 0.65 mol/Kg, more preferably between 0.30 mol/Kg and 0.60 mol/Kg, and most preferably between 0.35 mol/Kg and 0.55 mol/Kg in that when equilibrated at 45° C. with an aqueous solution containing 35% wt. dextrose and 15% wt. HCl, said organic phase is loaded to between 0.45 mol/Kg and 0.90 mol/Kg, preferably between 0.50 mol/Kg and 0.85 mol/Kg, more preferably between 0.55 mol/Kg and 0.80 mol/Kg, and most preferably between 0.60 mol/Kg and 0.75 mol/Kg and in that when equilibrated at 45° C. with an aqueous solution containing 35% wt. dextrose and 20% wt. HCl, said organic phase is loaded to between 0.55 mol/Kg and 1 mol/Kg, preferably between 0.60 mol/Kg and 0.95 mol/Kg, more preferably between 0.65 mol/Kg and 0.90 mol/Kg, and most preferably between 0.70 mol/Kg and 0.85 mol/Kg.

Said organic phase composition is preferably further characterized by an essentially linear distribution curve for HCl extraction from 35% wt. dextrose solution in a range between 1% wt. HCl and 20% wt. HCl.

Preferably said organic phase composition is characterized by a water concentration of between 2.0% and 7.0% when equilibrated at 45° C. with an aqueous solution containing 35% wt. dextrose and 10% wt HCl.

Preferably said organic phase composition is further characterized by an aqueous/organic phase separation time of less than 5 min as measure after shaking gently 50 times at 50° C. with an aqueous solution containing 35% wt. dextrose and 20% wt HCl.

In a further aspect of the present invention, there is provided a method for the recovery of HCl comprising
a) bringing an aqueous process stream comprising HCl and a solute, wherein HCl amount, concentration and purity are W1, C1 and P1, respectively, into contact at a temperature T1 with an organic phase according to any of claims 1-9, whereupon HCl selectively transfers to said organic phase to form an HCl-carrying extract; and
b) recovering, at a temperature T2, HCl from said HCl-carrying solvent to form a recovered HCl stream wherein HCl amount, concentration and purity are W2, C2 and P2, respectively, and a regenerated organic phase, wherein said recovering comprises at least one of
   b1. bringing said HCl-carrying extract into contact with an aqueous back-extracting stream, whereupon HCl transfers to said aqueous stream, and
   b2. distilling HCl from said HCl-carrying extract wherein
   (i) W2/W1 is greater than 0.5
   (ii) C2/C1 is greater than 0.7;
   (iii) P2/P1 is greater than 20; and
   (iv) T1 and T2 are less than 130° C.

In preferred embodiments of the present invention, said recovering comprises both bringing in contact and distilling and wherein said distilling precedes said bringing in contact.

Preferably, T2 is greater than T1 by at least 20° C.

Preferably, said solute is a carbohydrate, said carbohydrate concentration is greater than 15% wt. and said process stream is formed in a process of hydrolyzing a polysaccharide in a polysaccharide-containing material.

In preferred embodiments of the present invention, said method further comprises utilizing a step of using said recovered HCl stream for the hydrolysis of a polysaccharide.

In yet a further aspect of the present invention there is provided a method for the production of carbohydrate comprising
a. providing a feed comprising a polysaccharide;
b. hydrolyzing said polysaccharide in an HCl-comprising hydrolysis medium, wherein HCl amount, concentration and purity are W3, C3 and P3, respectively, to form a hydrolyzate comprising carbohydrate and HCl, wherein HCl amount, concentration and purity are W4, C4 and P4, respectively,
c. separating a portion of the HCl from said hydrolyzate to form a first separated HCl stream wherein HCl amount, concentration and purity are W5, C5 and P5, respectively, and an HCl-depleted hydrolyzate, wherein HCl amount, concentration and purity are W1, C1 and P1, respectively,
d. bringing said HCl-depleted hydrolyzate into contact at a temperature T1 with an organic phase according to the first aspect, whereupon HCl selectively transfers to said organic phase to form an HCl-carrying extract and an essentially HCl-free hydrolyzate;
e. recovering, at a temperature of T2, HCl from said HCl-carrying solvent to form a recovered HCl stream wherein HCl amount, concentration and purity are W2, C2 and P2, respectively, and a regenerated organic phase, wherein said recovering comprises at least one of
   e1. bringing said HCl-carrying extract into contact at a temperature T2 with an aqueous back-extracting stream, whereupon HCl transfers to said aqueous stream, and
   e2. distilling HCl from said HCl-carrying extract
f. combining at least a portion of said first separated HCl stream and a portion of said recovered HCl stream to form a reagent HCl stream, wherein HCl amount, concentration and purity are W6, C6 and P6, respectively, and
g. combining said reagent HCl stream with a lignocellulosic material to form said hydrolysis medium
wherein
   i. said first HCl stream is essentially carbohydrate free
   ii. W2/W1 is greater than 0.5; W1/W3 and W1/W4 are each less than 0.4, W5/W4 is greater than 0.1, and W6/W4 is greater than 1,
   iii. C2/C1 is greater than 0.7;
   iv. C3, C4, C5 and C6 are each greater than 30% wt;
   v. P2/P1 is greater than 20, and P3/P1 and P4/P1 are each greater than 50
   vi. P5 and P6 are each greater than 80% and
   vii. T1 and T2 are each less than 130° C.;

Preferable said recovering comprises both bringing in contact and distilling and said distilling precedes said bringing in contact.

In preferred embodiments of this aspect of the present invention, T2 is greater than T1 by at least 20° C.

In preferred embodiments of this aspect of the invention, the method further comprises a step (h) of separating another portion of HCl from said HCl-depleted hydrolyzate to form a second separated HCl stream wherein HCl amount, concentration and purity are W7, C7 and P7, respectively, and (i) combining at least a portion of said second separated HCl stream with at least a portion of said first separated HCl stream and a portion of said recovered HCl stream to form said reagent HCl stream, wherein W7/W4 is greater than 0.1, C7 is greater than 10% wt and P7 is greater than 80%.

In this embodiment, preferably C3 is greater than 36% wt, and C4, C5 and C6 are greater than 39% wt.

Preferably at least one of said first separated HCl stream or said recovered HCl stream is gaseous.

Preferably at least one of C1 and C7 is an azeotropic concentration. In preferred embodiments of this aspect of the invention said feed comprises a lignocellulosic material, wherein said hydrolyzing further forms an HCl-comprising lignin stream, wherein HCl amount, concentration and purity are W8, C8 and P8, respectively, wherein W8/W3 is greater than 0.2, C8 is greater than 35% and P8/P1 is greater than 20.

Preferably said method further comprises the steps of (j) separating HCl from said HCl-comprising lignin stream to form a third separated HCl stream wherein HCl amount, concentration and purity are W9, C9 and P9, respectively, and an HCl-depleted lignin stream and (k) combining at least a portion of said third separated HCl stream with at least a portion of said first separated HCl stream and a portion of said recovered HCl stream to form said reagent HCl stream, wherein C9 is greater than 30% wt, P9 is greater than 80% wt and W9/W8 is greater than 0.1.

Preferably said third stream is gaseous.

Preferably said method further comprises the steps of (I) separating HCl from said HCl-depleted lignin stream to form a fourth separated HCl stream wherein HCl amount, concentration and purity are W10, C10 and P10, respectively, and an essentially HCl-free lignin stream and wherein W10/W8 is greater than 0.1, C10 is greater than 10% wt and P10 is greater than 50% wt.

In preferred embodiments said separating HCl from said HCl-depleted lignin stream comprises a counter-current washing with water.

Preferably said separating HCl from said HCl-depleted lignin stream comprises distilling HCl in the presence of a first organic solvent.

Preferably said organic phase is an organic phase as defined above.

In preferred embodiments of this aspect of the present invention, said method further comprises a step (m) wherein an aqueous HCl solution is treated for water removal therefrom, wherein the ratio between the amount of removed water and W3 is smaller than 0.2, preferably smaller than 0.15, more preferably smaller than 0.1, and most preferably smaller than 0.05 and wherein C6 is greater than 39% wt.

In yet a further aspect of the present invention, there is provided a method for the production of a carbohydrate comprising
a. providing a lignocellulosic material feed comprising a polysaccharide;
b. hydrolyzing said polysaccharide in an HCl-comprising hydrolysis medium, wherein HCl amount, concentration and purity are W3, C3 and P3, respectively, to form a hydrolyzate comprising carbohydrate and HCl, wherein HCl amount, concentration and purity are W4, C4 and P4, respectively and an HCl-comprising lignin stream, wherein HCl amount, concentration and purity are W8, C8 and P8, respectively,
c. separating a portion of the HCl from said hydrolyzate to form a first separated HCl stream wherein HCl amount, concentration and purity are W5, C5 and P5, respectively, and an HCl-depleted hydrolyzate, wherein HCl amount, concentration and purity are W1, C1 and P1, respectively,
d. separating HCl from said HCl-comprising lignin stream to form a third separated HCl stream wherein HCl amount, concentration and purity are W9, C9 and P9, respectively, and an HCl-depleted lignin stream and.
e. bringing said HCl-depleted hydrolyzate into contact at a temperature T1 with an organic phase, selected from a group consisting of
   a. an organic phase as defined hereinbefore and whereupon HCl selectively transfers to said organic phase to form an HCl-carrying extract and an essentially HCl-free hydrolyzate;
f. recovering, at a temperature of T2, HCl from said HCl-carrying extract to form a recovered HCl stream, wherein HCl amount, concentration and purity are W2, C2 and P2, respectively, and a regenerated organic phase, wherein said recovering comprises at least one of
   f1. bringing said HCl-carrying extract into contact with an aqueous back-extracting stream, whereupon HCl transfers to said aqueous stream, and
   f2. distilling HCl from said HCl-carrying extract
g. combining at least a portion of said first separated HCl stream, a portion of said recovered HCl stream and a portion of said third separated HCl stream to form a reagent HCl stream, wherein HCl amount, concentration and purity are W6, C6 and P6, respectively, and
h. combining said reagent HCl stream with a lignocellulosic material to form said hydrolysis medium
   wherein
i. said first HCl stream is essentially carbohydrate free
ii. W2/W1 is greater than 0.5; W1/W3 and W1/W4 are each less than 0.4, W5/W4 is greater than 0.1, W6/W4 is greater than 1, W8/W3 is greater than 0.2 and W9/W8 is greater than 0.1
iii. C2/C1 is greater than 0.7;
iv. C3, C4, C5, C6, C8 and C9 are each greater than 30% wt;
v. P2/P1 and P8/P1 are each greater than 20 and, P3/P1 and P4/P1 are each greater than 50;
vi. P5, P6 and P9 are each greater than 80% and
vii. T1 and T2 are each less than 130° C.;
viii. each of said contacts comprises at least 3 counter-current stages.

Preferably said recovering comprises both bringing in contact and distilling and wherein said distilling precedes said bringing in contact.

In preferred embodiments of this aspect of the invention, T2 is greater than T1 by at least 20° C.

In preferred embodiments of this aspect of the present invention, said method further comprises the steps of (k) separating HCl from said HCl-depleted lignin stream to form a fourth separated HCl stream wherein HCl amount, concentration and purity are W10, C10 and P10, respectively, and an essentially HCl-free lignin stream and (I) combining at least a portion of said fourth separated HCl stream with at least a portion of said first separated HCl stream, a portion of said recovered HCl stream and a portion of said third separated HCl stream to form said reagent HCl stream, wherein W10/W8 is greater than 0.1, C10 is greater than 10% wt and P10 is greater than 50% wt.

In another preferred embodiment, said separating HCl from said HCl-depleted lignin stream comprises distilling HCl in the presence of a first organic solvent.

Preferably, said first organic solvent is essentially of the same composition as the solvent for said first component and for said second component.

Preferably C3, C4, C5, C6 and C9 are each greater than 39% wt.

Preferably at least one of said first separated HCl stream, said recovered HCl stream and said third separated HCl stream is gaseous.

Preferably at least one of C1 and C9 is of an azeotropic concentration.

In especially preferred embodiments of this aspect of the invention, said method further comprises a step (m) wherein an aqueous HCl solution is treated for water removal therefrom, wherein the ratio between the amount of removed water and W3 is smaller than 0.2, preferably smaller than 0.15, more preferably smaller than 0.1, and most preferably smaller than 0.05 and wherein C6 is greater than 39% wt.

Preferably said essentially HCl-free hydrolyzate comprises an organic impurity, further comprising a step of (n) contacting said essentially HCl-free hydrolyzate with a second organic solvent, whereupon said impurity transfers to said second organic phase to form an impurity-carrying organic phase and a purified essentially HCl-free hydrolyzate.

In other embodiments of the present invention, preferably said first organic solvent is essentially of the same composition as the solvent for said first component and for said second component.

In especially preferred embodiments of this aspect of the invention, said hydrolyzate further comprises an organic solute, and said method further comprises a step (o) of bringing said hydrolyzate into contact at a temperature T3 with a third organic solvent, whereupon said organic solute selectively transfers to said third organic solvent to form an organic solute-depleted hydrolyzate and a first organic solute-carrying solvent and; optionally, (p) recovering said third solvent and organic solute from said first extractives-carrying solvent to form separated organic solute and regenerated third solvent.

In especially preferred embodiments of this aspect of the invention, said lignin stream comprises an organic solute, and said method further comprises a step of (q) bringing said lignin stream into contact, at a temperature T4, with a fourth organic solvent, whereupon said organic solute selectively transfers to said fourth organic solvent to form an organic solute-depleted lignin stream and a second organic solute-carrying solvent and; optionally, (r) recovering said fourth solvent and organic solute from said second organic solute-carrying solvent to form a separated organic solute and a regenerated fourth solvent.

In preferred embodiments of this aspect of the invention, said essentially HCl-free lignin stream comprises an organic solute, and said process further comprises the step of (s) bringing said essentially HCl-free lignin stream into contact at a temperature T4 with a fifth organic solvent, whereupon said organic solute selectively transfers to said fifth organic solvent to form an organic solute-depleted essentially HCl-free lignin stream and a third organic solute-carrying solvent and; optionally, (t) recovering said fifth solvent and organic solute from said third organic solute-carrying solvent to form a separated organic solute and a regenerated fifth solvent.

Preferably said organic solute is a wood extractive.

In preferred embodiments of this aspect of the present invention, at least one of said third organic solvent, fourth organic solvent and fifth organic solvent is essentially of the same composition as the solvent for the amine and the organic acid.

In other preferred embodiments of this aspect of the present invention, at least one of said third organic solvent, fourth organic solvent and fifth organic solvent is essentially of the same composition as the solvent for said first component and for said second component.

The invention will now be described in connection with certain preferred embodiments with reference to the following illustrative figure so that it may be more fully understood.

With specific reference now to the figure in detail, it is stressed that the particulars shown are by way of example and for purposes of illustrative discussion of the preferred embodiments of the present invention only and are presented in the cause of providing what is believed to be the most useful and readily understood description of the principles and conceptual aspects of one of the methods of the invention. In this regard, no attempt is made to show details of the invention in more detail than is necessary for a fundamental understanding of the invention, the description taken with the attached figure making apparent to those skilled in the art how the several forms of the invention may be embodied in practice.

In the drawings:

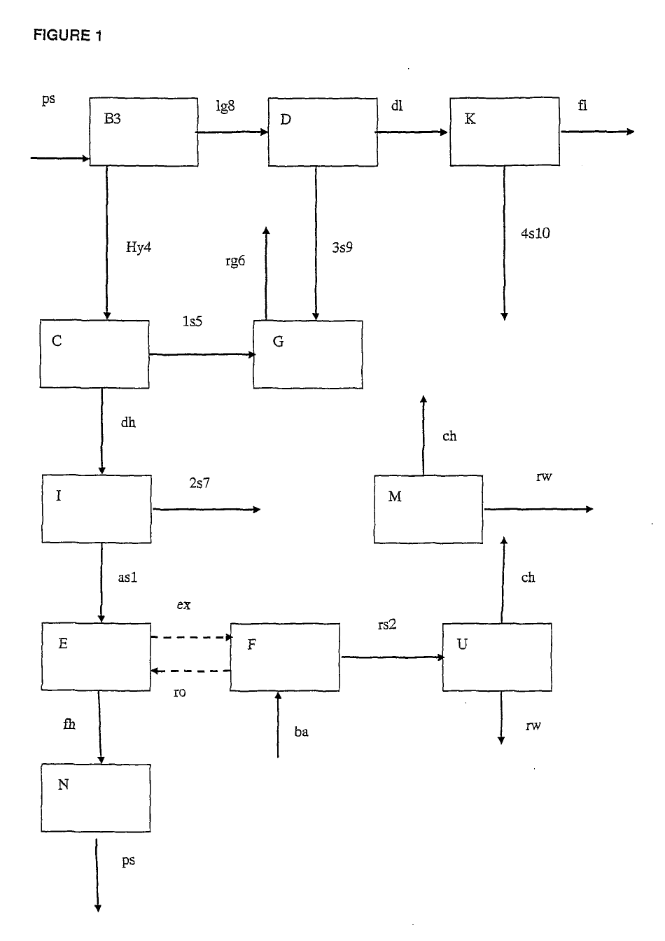

FIG. 1 is a flow diagram of a preferred process of the present invention.

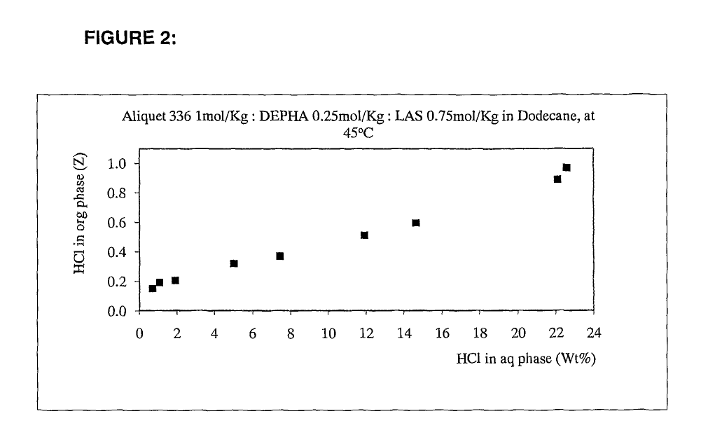

FIG. 2 is a graph presenting the results of HCl distribution between phases in a preferred embodiment of the invention.

### DETAILED DESCRIPTION

Preferred embodiments of the present invention are described in the following in reference to the flow diagram in FIG. 1. In the following, numbers and letters in [X] refer to operations (boxes in the diagram) and ones in <X> refer to streams (arrows).

According to the method of the present invention, a polysaccharide in a polysaccharide-comprising feed (<ps> in FIG. 1) is hydrolyzed in an HCl-comprising hydrolysis medium (hydrolysis takes place in [B3]). Such medium is formed, according to an embodiment, by contacting said feed with a reagent HCl stream <rg6>. According to one embodiment, that contacting operates in a batch mode, while according to another it is continuous. According to a preferred embodiment, contacting is conducted counter-currently, e.g. in a tower reactor into which the feed is introduced from the top and the reagent HCl stream flows in from the bottom. The reagent HCl stream comes in containing essentially no carbohydrates. As it flows upwards, carbohydrates from polysaccharides' hydrolysis start to build up in that stream reaching concentrations greater than 15, preferably greater than 20%, more preferably greater than 25%, and most preferably greater than 30%. At the same time, the lignocellulosic material losses its polysaccharides as it moves downwards, counter-currently to the reagent HCl stream. According to another embodiment, the lignocellulosic material is fed into a series of reactors and the reagent HCl stream moves into one, then out of it and into the next one, without moving the remaining, solid, polysaccharides-depleted lignocellulosic material from one reactor to the other. In that way, the lignocellulosic material is first contacted with an aqueous HCl solution comprising carbohydrates from previous stages. As its residence time increases, it is brought in contact with an HCl solution containing less and less carbohydrates.

Various polysaccharide-comprising feeds can be treated according to the method of the present invention. The terms saccharide, sugar and carbohydrates are used here interchangeably. Any polysaccharide is suitable, e.g. polymers of glucose, xylose, arabinose, and mannose, etc. Most of the sugars of interest are either 6 carbon sugars (hexoses) or 5 carbon sugars (pentoses). The terms glucose and dextrose are used here interchangeably. The polymers could be of a single sugar or comprise multiple carbohydrates, e.g. hemicellulose consisting mainly of xylose and arabinose or glucomannane consisting of glucose and mannose. Various polysaccharides are suitable for the method of the present invention. Of particular interest are cellulose and hemicellulose.

Any polysaccharide-comprising feed is suitable, particularly ones that comprise cellulose, e.g. recycled paper, co-products of the pulp and paper industry and biomass cell walls. Of particular interest are lignocellulosic materials. As used here, the term lignocellulosic material refers to any material comprising cellulose and lignin. Typically, lignocellulosic material further comprises hemicellulose and additional components such as extractives and mineral compounds. The weight ratios between the various components—mainly the three major ones, i.e. cellulose, hemicellulose and lignin—change according to the source of the lignocellulosic material. The same is true for the mineral compounds, also referred to as ashes.

The term extractives, as used here, means oil-soluble compounds present in various lignocellulosic feeds or products of their conversion (e.g. hydrolysis).

Various lignocellulosic materials are known and are suitable for the present invention. Of particular interest are wood chips from the construction board industry, agricultural wastes, such as stover and corn cobs and energy crops. Lignocellulosic material could be used as such or after some pre-treatment. Any pre-treatment that does not lead to the hydrolysis of the majority of the cellulose content is suitable.

According to an embodiment, the lignocellulosic material is dried prior to said combining with said reagent HCl stream. Lignocellulosic material could be obtained from various sources at various degrees of moisture. Various methods of drying are suitable.

According to another embodiment, the lignocellulosic material is comminuted prior to said combining with said reagent HCl stream.

According to one embodiment, the polysaccharides of the lignocellulosic material are not hydrolyzed prior to said combining with said reagent HCl stream. According to another embodiment, the lignocellulosic material is pre-treated for the removal and/or hydrolysis of hemicellulose prior to said combining with said reagent HCl stream. Such removal and/or hydrolysis could be conducted by various means, e.g. elevated temperature treatment with water/steam and/or dilute acid. Such elevated temperature pre-treatment extracts hemicellulose into an aqueous phase, hydrolyzes hemicellulose into water soluble sugars and combinations thereof, leading to lignocellulosic material in which cellulose is the main polysaccharide.

According to other embodiments, the lignocellulosic material is treated by at least one of steam explosion, ammonia explosion and delignification.

The hydrolysis medium comprises, at least initially, HCl in an amount, concentration and purity of W3, C3 and P3, respectively. In a preferred embodiment, the hydrolysis of the present method is continuous and the amount is presented in terms of flow rate, e.g. as the ratio between the flow rate of the acid and that of the initial polysaccharide-comprising feed in said medium. According to a preferred embodiment, that ratio is between 0.2 and 5 (w/w), preferably between 0.5 and 3 (w/w).

Unless specified otherwise, the concentration of a component in a medium (e.g. gaseous stream, a solution or a suspension) is presented in weight percent (% wt) calculated from the weight of said component in that medium and the combined weights of that component and the water in that medium. Thus, e.g. in a medium composed of 30 Kg water, 20 Kg of HCl and 50 Kg of carbohydrate, the concentration of HCl according to the presentation herein is 40%. According to a preferred embodiment, C3 is greater than 36%, more preferably greater than 39% and most preferably greater than 40%.

Unless specified otherwise, the purity of a component in a medium is the purity in a homogeneous phase (liquid or gas). In case the medium comprises insolubles, the purity referred to is that in the solution that would form on separation of those insolubles. Unless specified otherwise, the purity is calculated on a water-free and weight basis. As used herein, purity means the ratio between the wt % concentration of HCl and the wt % concentration of other solutes combined. Thus, HCl purity in a solution composed of 50 Kg water, 20 Kg of HCl and 20 Kg of carbohydrate and 10 Kg mineral salt, as presented herein, is 40%/60% i.e. 0.67.

According to the method of the present invention, hydrolysis of the polysaccharide takes place in B3. According to the embodiment wherein the lignocellulosic material undergoes pre-hydrolysis, hydrolysis in B3 is mainly of cellulose. In case there is no pre-hydrolysis, both hemicellulose and cellulose are hydrolyzed. HCl acts as a catalyst and is not consumed, except possibly for neutralizing basic components of the lignocellulosic material. The amount of solids decreases due to the hydrolysis so that the acid to solids ratio increases. According to an embodiment of the invention, at least 70% wt of the polysaccharide in the feed hydrolyzes into soluble carbohydrates, preferably more than 80%, more preferably more than 90%, and most preferably more than 95%. As a result, the concentration of the soluble carbohydrates in the medium increases with the progress of the hydrolysis reaction.

The hydrolysis in B3 forms a hydrolyzate <hy4> comprising carbohydrate and HCl, wherein HCl amount, concentration and purity are W4, C4 and P4, respectively. Preferably, said hydrolyzate is essentially solids free. In case of hydrolyzing a lignocellulosic material feed comprising insoluble compounds, such as lignin in lignocellulosic material, those are preferably separated to form a stream comprising those solids, e.g. a lignin stream <Ig8> as further described in the following.

While there is no significant consumption of HCl in the hydrolysis process, W4 is in many cases smaller than W3, e.g. in the case shown in FIG. 1, since part of the acid is contained in <Ig8>. C4 is similar in size to C3. As carbohydrates are being added into the solution during the hydrolysis, the purity of the acid in the solution decreases. According to various embodiments, P4 is between 20% and 70%, more preferably between 30% and 60%.

According to an embodiment, the lignocellulosic feed further comprises an organic solute, e.g. tall oil, and a fraction of the organic solute is dissolved in the formed hydrolyzate. According to a related embodiment, the organic-solute-comprising hydrolyzate is brought into contact at a temperature T3 with a third organic solvent, whereupon said organic solute selectively transfers to said third organic solvent to form an organic solute-depleted hydrolyzate and a first organic solute-carrying solvent. According to an embodiment, the organic solute-carrying solvent has a commercial value as such. According to another embodiment, the method further comprises a step of recovering said third solvent and organic solute from said first extractives-carrying solvent to form a separated organic solute and a regenerated third solvent. Various methods are suitable for such recovering, including distilling the third organic solvent and extracting it into another solvent, wherein the organic solute has limited miscibility. According to an embodiment, said organic solute is a tall oil.

Tall oils are produced in various industries, and are used for various applications.

The present invention further provides a method for the production of wood extractives comprising (a) providing a feed comprising a polysaccharide and extractives; (b) hydrolyzing said polysaccharide in an HCl-comprising hydrolysis medium, wherein HCl concentration is C3, to form a hydrolyzate comprising carbohydrate, extractives and HCl, wherein extractives to HCl ratio is R¹_{E/A} and wherein extractives to carbohydrate ratio is R¹_{E/C}; (c) optionally, treating said hydrolyzate to form treated hydrolyzate (d) bringing said hydrolyzate or treated hydrolyzate into contact at a temperature T3 with a third organic solvent, whereupon extractives selectively transfer to said third organic solvent to form an extractives-depleted hydrolyzate and a first extractives-carrying solvent and; wherein extractives to HCl ratio is R²_{E/A} and wherein extractives to carbohydrate ratio is R²_{E/C}; and (e) optionally, recovering said third solvent and extractives from said first extractives-carrying solvent to form separated extractives and regenerated third solvent. According to an embodiment of the invention, said contacting with the third organic solvent not only separates the extractives from the hydrolyzate, but also contributes to their purification. Thus, according to an embodiment, R²_{E/A} is at least 10 folds greater than R¹_{E/A}, more preferably at least 50 folds greater and most preferably at least 100 folds greater. According to another embodiment, R²_{E/C} is at least 10 folds greater than R¹_{E/C}, more preferably at least 50 folds greater and most preferably at least 100 folds greater.

According to a preferred embodiment, said contacting of the hydrolyzate with the third organic solvent is conducted while the hydrolyzate is high in acid concentration, e.g. while the acid concentration therein is at least 25%, preferably at least 28% and more preferably at least 32%. According to a related embodiment, said contacting is conducted prior to the following step of separating a portion of the HCl from the hydrolyzate. The inventors have found that the solubility of some of those organic solutes in the hydrolyzate decreases with decreasing HCl concentration. Contacting with the third organic solvent, while HCl concentration is still high, provides for high yield of recovering organic solutes on the one hand and avoids their precipitation in the next step, which precipitation may form undesired coating of equipment.

According to another preferred embodiment, the third organic solvent is essentially of the same composition as the solvent for the amine and the organic acid (the third component) of the organic phase composition. According to still another preferred embodiment, that third organic solvent is essentially of the same composition as the solvent for said first component and for said second component (i.e. the third component). As used herein, the term of essentially the same composition for two components means that the two are composed of the same compound in case each of those is composed of a single compound, or, in case of mixtures, that at least 50% wt. of the composition of one component is identical to at least 50% wt. of the composition of the other component. That is, for example the case wherein the two components are mixtures of hydrocarbons (e.g. C6 to C9 ones) and wherein at least 50% wt. of each mixture is the same hydrocarbon, e.g. heptane. According to a preferred embodiment, the thirds organic solvent is selected from the group consisting of heptanes, octanes and nonanes, and most preferably heptanes.

According to a preferred embodiment, said contacting with said third organic solvent is conducted at a temperature T3 that is less than 120° C., more preferably less than 100° C. and most preferably less than 80° C.

The method of the presented invention further comprises a step [C] of separating a portion of the HCl from said hydrolyzate to form a first separated HCl stream <1 s5>, wherein HCl amount, concentration and purity are W5, C5 and P5, respectively, and an HCl-depleted hydrolyzate <dh>. According to a preferred embodiment, said separation involves distilling HCl out of the hydrolyzate and the first separated stream <1 s5> is gaseous. Preferably, a significant fraction of the acid in the hydrolyzate is distilled out in [C], so that W5/W4 is at least 0.2, preferably at least 0.3 and more preferably at least 0.4. Said first separated HCl stream may contain small amounts of water, e.g. water vapors in a gaseous HCl stream, and possibly also small amounts of some other volatile components of the hydrolyzate. Yet, both C5 and P5 are high, typically greater than 90%, preferably greater than 95% and more preferably greater than 97%.

According to an embodiment, the method further comprises a step [I] of separating another portion of HCl from said HCl-depleted hydrolyzate to form a second separated HCl stream <2s7>, wherein HCl amount, concentration and purity are W7, C7 and P7, respectively, and a further-depleted hydrolyzate (<as1> in FIG. 1). According to a preferred embodiment, said separation in [I] involves distilling HCl out of the hydrolyzate and the second separated stream is gaseous. Preferably, a significant fraction of the acid in the HCl-depleted hydrolyzate is distilled out in [I], so that W7/W4 is at least 0.1, preferably at least 0.2 and more preferably at least 0.3. Said second separated HCl is, according to a preferred embodiment a water-HCl azeotrope so that C7 is about azeotropic. <2s7> is essentially carbohydrates free, but may contain small amounts of volatile components of the hydrolyzate. Yet, P7 is high, typically greater than 90%, preferably greater than 95% and more preferably greater than 97%. According to an embodiment of the invention, at least a fraction of the second separated HCl stream is further treated for concentration (or water removal) as explained below in more details. Said high purity, particularly the fact that the second separated stream is essentially free of carbohydrates, presents an important advantage in such further concentration.

HCl amount, concentration and purity in the HCl-depleted hydrolyzate <dh>, formed after separating the first separated HCl stream, or in <as1>, formed after separating the second separated HCl stream (where implemented), are W1, C1 and P1, respectively. According to a preferred embodiment, the amount of acid in that stream (W1) is small compared with the amount of acid in hydrolysis medium (W3) and with the amount of acid in the hydrolyzate stream (W4). Thus, according to the method of the present invention, W1/W3 and W1/W4 are each smaller than 0.4, preferably smaller than 0.3 and more preferably smaller than 0.25.

According to an embodiment, of the present invention, carbohydrates concentration in the HCl-depleted hydrolyzate is about the same as in the hydrolyzate. Yet, carbohydrates concentration in the further-depleted hydrolyzate is greater, e.g. greater than 30% wt, preferably greater than 40% wt, more preferably greater than 50% wt and most preferably greater than 55% wt.

According to the method of the present invention, the HCl-depleted hydrolyzate <dh> or a stream formed by further treating it (e.g. the further-depleted hydrolyzate formed on separating the second separated HCl stream, <as1>) is brought into contact [E] at a temperature T1 with an organic phase (<ro> in FIG. 1), whereupon HCl selectively transfers to said organic phase to form an HCl-carrying extract <ex> and an essentially HCl-free hydrolyzate <fh>. Contacting in [E] is conducted counter-currently and preferably involves at least three contact stages, preferably at least four. Any liquid-liquid contactors are suitable for the purpose of the contacting of the present invention, including commercially available mixer-settlers, columns, pulsating columns and centrifugal contactors. According to various embodiments, contacting is conducted at atmospheric pressure and T1 is lower than 130° C., preferably lower than the boiling temperature of the various streams. According to a preferred embodiment, T1 is lower than 90° C., more preferably lower than 70° C. and most preferably lower than 60° C.

According to an embodiment of the method, the organic phase is contacted with the HCl-depleted hydrolyzate formed after separating the first separated HCl stream from the hydrolyzate. According to another embodiment, it is contacted with the further-depleted hydrolyzate formed on separating the second separated HCl stream. According to alternative embodiments, contacting is with at least one of those streams after some additional treatment. Such additional treatment involves, according to an embodiment, combining with at least one other aqueous HCl solution, whether comprising carbohydrates or not. Thus, according to an embodiment, combining is with the fourth separated HCl stream. The latter is mixed with the HCl-depleted hydrolyzate or the further-depleted hydrolyzate prior to contacting with the organic phase according to one embodiment. According to another embodiment, that hydrolyzate is first contacted with the organic phase in a multiple-stage, counter-current operation wherein the HCl concentration changes from one step to the other and the other aqueous HCl solution is introduced in one of those stages, preferably at a stage where the HCl concentrations of the two streams are similar.

On such contacting with the organic phase, HCl selectively transfers from the hydrolyzate into the organic phase to form an HCl-carrying extract and an essentially HCl-free hydrolyzate <fh>. As used herein, essentially HCl-free means having low HCl content, e.g. lower than 2%, preferably lower than 1%, more preferably lower than 0.5% and most preferably lower than 0.2%. Reaching these low HCl concentrations, in the essentially free hydrolyzate, represents high yield of acid recovery from the hydrolyzate. Thus, according to an embodiment of the method, at least 95% of the acid in the hydrolyzate is extracted (i.e. transferred into the organic phase), more preferably at least 96% and most preferably at least 98%.

The essentially-HCl-free hydrolyzate comprises carbohydrate products of the polysaccharides' hydrolysis. According to an embodiment, the carbohydrates are of low degree of polymerization, e.g. monosaccharides, disaccharides and oligosaccharides (e.g. trimers and tetramers) at various ratios depending on the parameters of the hydrolysis reaction (such as HCl concentration and residence time) and on the methods used for the separation of the first separated HCl stream (and the second where applicable). According to an embodiment, the carbohydrate concentration in the essentially-HCl-free hydrolyzate is greater than 25% wt, preferably greater than 35% wt, more preferably greater than 40% wt. According to an embodiment, the carbohydrates concentration there is greater than 60%.

According to an embodiment, the essentially-HCl-free hydrolyzate is contacted with a second organic solvent. According to an embodiment, the essentially-HCl-free hydrolyzate comprises an organic compound and said organic compound is selectively transferred into the second organic solvent to form an organic solution carrying that organic compound. According to an embodiment, the formed organic solution is treated for the separation of the organic compound from the second organic phase to form a separated organic compound and a regenerated second organic solvent.

According to an embodiment, that organic compound in said essentially HCl-free hydrolyzate comprises components of the organic phase, e.g. an amine, an organic acid or their combination. Such components of the organic phase are found in the essentially-HCl-free hydrolyzate due to some (very limited) dissolution and/or due to entrainment of the organic phase therein during the contacting step. According to an embodiment, the second organic solvent is essentially of the same composition as the solvent for the amine and the organic acid. According to another embodiment, the second organic solvent is essentially of the same composition as the solvent for said first component and for said second component (i.e. the third component). According to an embodiment, the organic compound comprises at least one component of the organic phase and the organic solution carrying that organic compound is combined with the organic phase as such or after some pre-treatment, e.g. partial distillation of the second organic solvent.

According to another embodiment, the organic compound in said essentially HCl-free hydrolyzate is an organic impurity and contacting forms an impurity-carrying organic phase and a purified, essentially HCl-free hydrolyzate.

According to an embodiment, the second organic solvent is selected from the group consisting of aliphatic and aromatic hydrocarbons. Preferably, those hydrocarbons are of five to ten carbon atoms, more preferably six to nine carbon atoms. According to a preferred embodiment, the second organic solvent is selected from the group consisting heptanes and octanes (both linear and branched ones).

The essentially-HCl-free hydrolyzate is suitable, as such or after an additional modification, for use in various applications. Of particular interest are bioconversion and chemical conversion into products such as biofuels, and chemicals. Bioconversion typically involves fermentation to form fermentation products, while chemical conversion uses catalysts of various types. As indicated above, the essentially-HCl-free hydrolyzate comprises, according to various embodiments, oligosaccharides, typically in a mixture with monosaccharides. Some of those biological and chemical conversions handle oligomers very well. Others may require a lower content of oligomers or conversion of larger oligomers into shorter ones.

According to an embodiment of the invention, the carbohydrate concentration in the HCl-depleted hydrolyzate and/or the further HCl depleted hydrolyzate is decreased by combining with a process stream that is lower on carbohydrate and preferably quite high in HCl concentration. One example for that process stream is the fourth separated HCl stream from the treatment of the HCl-depleted lignin stream. According to an embodiment, at least a fraction of said process stream, e.g. the fourth separated HCl stream, is combined with the HCl-depleted hydrolyzate to form a first combined stream. According to an embodiment, that first combined stream is then treated for the separation of the second separated HCl stream and such treatment provides for the hydrolysis of oligomers in the HCl-depleted hydrolyzate.

According to an embodiment, alternatively or in addition, hydrolysis of the oligomers into monomers, dimers and/or shorter oligomers is conducted on the essentially-HCl-free hydrolyzate after said contacting with the organic phase. According to one embodiment, such hydrolysis is chemically catalyzed, e.g. acid catalyzed. According to another, it is conducted enzymatically, using suitable enzymes, e.g. ones developed for the hydrolysis of cellulose and/or hemicellulose. According to still another embodiment, both chemical and biologic catalysis are used.

According to still another embodiment, chemical catalysis is used for the hydrolysis of said oligomers and said hydrolysis is conducted during the contacting with the organic phase. According to a preferred embodiment, the HCl-depleted hydrolyzate, the further HCl-depleted hydrolyzate or their combinations with other streams is contacted with the organic phase counter-currently in a multiple-step operation. Moving from one step to the other, the concentration of the HCl in the aqueous solution decreases, while the carbohydrates concentration therein stays about the same. Before reaching essentially full removal of the acid, when the acid concentration in the aqueous phase is suitable, the aqueous solution is kept at conditions facilitating the hydrolysis of the oligomers therein to form a monomers-enriched solution. According to an embodiment, the suitable concentration of the HCl is in the range between 0.5% wt and 5% wt. According to an embodiment, the conditions for facilitating the hydrolysis involve according to various embodiments a temperature in the range between 50° C. and 130° C., and a residence time between 1 min and 60 min. The formed monomers-enriched solution is then returned to the next stage of contacting with the organic phase for the completion of HCl extraction.

According to an embodiment, for such chemical or biological hydrolysis, the hydrolyzate is diluted and optionally re-concentrated. According to a preferred embodiment, such re-concentration is conducted after the catalyst is removed.

According to a preferred embodiment, the organic phase is a regenerated organic phase from a previous stage, e.g. a back-extraction stage or a distillation stage.

According to an embodiment of this third aspect, the organic phase is a substantially water-immiscible extractant comprising: (a) an oil-soluble amine, which amine is substantially water-insoluble, in both free and salt forms; (b) an oil soluble organic acid which acid is substantially water insoluble both in free and in salt form; and (c) a solvent for the amine and organic acid;

As used here, the term “in salt form” when referring to amines means when protonated (or when in quaternary form). The term “in salt form” when referring to organic acids means when dissociated. Typically both organic amines and acids, when in salt form, have higher solubility in water compared with the same amine or acid in a free form. The solubility in water of the organic acids and amines of the present invention (in both free and salt form) is typically less than 2%, preferably less than 1%, more preferably less than 0.5% and most preferably less than 0.1%.

According to an embodiment, the composition of the organic phase is that of the extractant in IL 189699.

According to an embodiment, the oil-soluble amine is selected from a group consisting of primary, secondary, tertiary, quaternary amines and their mixtures, characterized by having at least 10, preferably at least 14, carbon atoms. Examples of commercially available suitable amines are Primene JM-5, and Primene JM-T (which are primary aliphatic amines in which the nitrogen atom is bonded directly to a tertiary carbon atom) sold by Rohm and Haas Chemical Co.; Amberlite LA-1 and Amberlite LA-2, which are secondary amines sold by Rohm and Haas; Alamine 336, a tertiary tricaprylyl amine (TCA) and Alamine 304, a tertiary trilaurylamine (TLA), both sold by Cognis, tris-2-ethylhexyl amine (TEHA) sold by BASF and Aliquate 336, a quaternary tricaprylyl methyl amine sold by Cognis.

According to an embodiment, the oil-soluble organic acid is selected from the group consisting of aliphatic and aromatic sulfonic acids and alpha-, beta- and gamma-chloro and bromo substituted carboxylic acids, e.g., hexadecylsulfonic acid, didodecylnaphthalene disulfonic acid, alpha-bromo lauric acid, beta-, beta-dichloro decanoic acid and gamma dibromo octanoic acid, etc. and organic acids with at least 6, preferably at least 8, and most preferably at least 10, carbon atoms, such as capric acid.

The solvent for the amine and organic acid can be chosen from a wide range of organic liquids known to persons skilled in the art which provide for greater ease in handling and extracting control. Said carrier solvents can be unsubstituted or substituted hydrocarbons in which the organic acid and amine are known to be soluble and which are substantially water-insoluble, e.g., kerosene, mineral spirits, naphtha, benzene, xylene, toluene, nitrobenzene, carbon tetrachloride, chloroform, trichloroethylene, etc. Also higher oxygenated compounds such as alcohols, ketones, esters, ethers, etc., that may confer better homogeneity and fluidity and others that are not acids or amines, but which may confer an operationally useful characteristic, can also be included. According to an embodiment, the solvent for the amine and organic acid is essentially of the same composition as at least one of said first organic solvent, said second organic solvent, said third organic solvent, said fourth organic solvent and said fifth organic solvent.

According to a preferred embodiment, the organic phase contacted with the HCl-depleted hydrolyzate, the further HCl-depleted hydrolyzate and combinations containing those streams comprise (a) a first component selected from the group consisting of quaternary amines, (b) a second component selected from (b1) the group consisting of category B organic acids (b2) the group consisting of a mixtures of category B organic acids and category C organic acids at a B/C molar ratio of R_{B/C} and (b3) the group consisting of a mixtures of category A organic acids and category C organic acids at an A/C molar ratio of R_{A/C} and (c) a third component selected from the group consisting solvents for said first component and for said second component, wherein (i) all three components are oil-soluble and water-insoluble (ii) the molar concentration of each of said first component and said second component is greater than 0.6 mol/Kg; (iii) the molar ratio between said second component and said first component is greater than 0.9; (iv) R_{B/C} and R_{A/C} are greater than 2; (v) category A organic acids are selected from the group consisting of poly-aromatic sulfonic acids, and naphthalene sulfonic acids and acids with pKa in the range within +/−0.5 pKa units of that of naphthalene sulfonic acid; (vi) category B organic acids are selected from the group consisting of mono-aromatic sulfonic acids, and benzene sulfonic acids and acids with pKa in the range within +/−0.5 pKa units of that of benzene sulfonic acid, and (vii) category C organic acids are selected from the group consisting of phosphoric acid esters and acids with pKa in the range within +/−0.5 pKa units of that of di-octyl esters of phosphoric acid.

According to an embodiment, the third component, selected from the group consisting of solvents for said first component and for said second component, is essentially of the same composition as at least one of said first organic solvent, said second organic solvent, said third organic solvent, said fourth organic solvent and said fifth organic solvent. According to another embodiment said third component is composed of at least 70% wt. hydrocarbon and said hydrocarbon is an aliphatic hydrocarbon, an aromatic hydrocarbon or a combination thereof.

The method of the present invention further comprises a step ([F] in FIG. 1) of recovering, at a temperature of T2, HCl from said HCl-carrying extract to form a recovered HCl stream wherein HCl amount, concentration and purity are W2, C2 and P2, respectively, and a regenerated organic phase, wherein said recovering comprises at least one of (i) bringing said HCl-carrying extract into contact with an aqueous back-extracting stream, whereupon HCl transfers to said aqueous stream, and (ii) distilling HCl from said HCl-carrying extract

The HCl-carrying extract is introduced to recovering as such (i.e. as formed in [E]) or after some pre-treatment. Such pre-treatment may include operations known in the industry, such as washing with a small amount of water in order to remove entrained aqueous solution and heating. According to an embodiment of the invention, the composition of that extract is modified prior to said contacting, e.g. by the addition or the removal of an extraction modifier. An extraction modifier is a compound that affects the degree of extraction, e.g. by affecting the basicity of the amine component of the extractant. According to one example, a volatile alcohol, such as pentanol, is present in the extractant during the extraction in [E], but is removed from the extract prior to said step of recovering.

Thus, according to one embodiment, said HCl-carrying extract <ex> is brought in contact at a temperature T2 with an aqueous back-extracting stream <ba>, whereupon HCl transfers to said aqueous stream to form a recovered HCl stream <rs2>, wherein HCl amount, concentration and purity are W2, C2 and P2, respectively, and a regenerated organic phase. The step of bringing said HCl-carrying extract in contact with an aqueous back-extracting stream is also referred to as back-extraction and the recovered HCl stream is also referred to as back-extract. Contacting is conducted counter-currently and involves at least three contact stages, preferably at least four. Any liquid-liquid contactors are suitable for the purpose of the present invention, including commercially available mixer-settlers, columns, pulsating columns and centrifugal contactors.

Water and aqueous solutions could be used as the aqueous back-extracting stream. According to an embodiment, the aqueous back-extracting stream is a process stream formed in another part of the process, including dilute HCl solutions. According to a preferred embodiment, the temperature of the back-extraction (T2) is greater than that of extraction (T1), preferably by at least 10° C., more preferably at least 20° C.

According to another embodiment, recovery comprises distilling HCl from said HCl-carrying extract, whereby a gaseous HCl stream is formed. Such distilling is conducted, according to a preferred embodiment, at a temperature lower than 160° C., more preferably lower than 140° C., and most preferably lower than 120°. According to various embodiments, such distilling is conducted under vacuum and/or with the aid of a carrier gas, e.g. steam or a hydrocarbon.

According to still another embodiment, recovery comprises both distilling and back-extraction. According to a related embodiment, distilling is conducted first and then the back-extraction.

In case of recovering by distilling, the formed gaseous HCl stream, or a product of its condensation, is referred to as the recovered HCl stream. In case of back-extraction, the back-extract is referred to as the recovered HCl stream. In case recovery comprises both distilling and back-extraction, the gaseous phase, the back-extract or their combination is referred to as the recovered HCl stream. In such latter case, W2, C2 and P2 are referred to the combination of those streams, even if they are not combined in the process.

According to an important embodiment of the invention, the preferred extractant allows for high reversibility of the extraction so that essentially all the extracted HCl in recovered in the recovered HCl stream <rs2>, e.g. more than 80%, preferably more than 90%, more preferably more than 95% and most preferably more than 98%. According to an embodiment, W2/W1 is greater than 0.9, more preferably greater than 0.94 and most preferably greater than 0.98.

According to an embodiment, the extractant out of the recovering step is used again (as <ro>) in HCl extraction in [E] as such or after some pretreatment. Such pretreatment could be selected from ones common to solvent-extraction based industries, e.g. removal of accumulating impurities. According to one embodiment, such removal is conducted by contacting the regenerated organic phase with an alkali solution. According to a preferred embodiment, only a fraction of the regenerated organic phase is treated in each cycle.

As indicated, the preferred organic phase of the present invention provides for reversible extraction, which means that not only essentially all the extracted acid is recovered from the HCl-carrying extractant, but that the recovered HCl stream is of a relatively high concentration, including in recovery by back-extraction. Differently put, in back-extraction, the ratio between the aqueous back-extracting stream and the HCl-carrying organic phase is such that the concentration in the recovered HCl stream is not much lower than that in the aqueous feed to the extraction, or even higher. Thus, according to an embodiment of the invention, C2/C1 is greater than 0.6, preferably greater than 0.7, more preferably greater than 0.8 and most preferably greater than 0.9. In case of recovering, at least partially by distilling, the recovered HCl stream is at least partially a gaseous stream highly concentrated in HCl.

The organic phase (extractant) of the present invention is highly selective to HCl over other solutes in the aqueous solution brought in contact with it in the extraction step [E]. For example, there is essentially no carbohydrates co-extraction with the acid. The HCl-carrying extractant carries very little other components than those of the organic phase, HCl and some water. Since in the recovery step [F] no impurities are added, the recovered HCl stream is much purer than in the stream entering the extraction and P2/P1 is greater than 20, preferably greater than 30, more preferably greater than 50 and most preferably greater than 70.

In a preferred embodiment the recovered HCl stream is of a concentration lower than azeotropic and is concentrated [U] to azeotropic concentration.

In yet another preferred embodiment, the recoverd HCl stream is of a concentration higher than azeotropic and is evaporated [U] to generate azeotropic HCl and gaseous HCl.

According to the method, at least a portion of said first separated HCl stream, at least a portion of said third separated HCl stream and at least a portion of said recovered HCl stream are combined to form a reagent HCl stream. Said first separated HCl stream, said third separated stream, said recovered HCl stream and their portions could be combined as such or after some additional treatment. Thus, according to the embodiment wherein the recovered stream is obtained at sub-azeotropic concentration and wherein said recovered stream is concentrated to form an azeotropic concentration, said formed azeotropic solution could be used to form that reagent HCl. According to various embodiments, also other HCl-comprising streams are combined, e.g. at least a portion of the second separated HCl stream and at least a portion of the fourth recovered HCl stream. According to still another embodiment, those streams are combined indirectly. According to a specific case, said second separated HCl stream, a portion of it, said recovered HCl stream, a portion of it, products of their conversion and various combinations of those, is used to form said third recovered HCl, said fourth recovered HCl stream or both, becomes a part of said thirds or fourth recovered stream and is used as such to form said reagent stream. The amount, concentration and purity of HCl in said reagent stream are W6, C6 and P6, respectively. According to the method of the present invention, said reagent stream is used to form hydrolysis medium.

In preferred embodiments of this aspect of the invention, C3, C4, C5 and C6 are each greater than 38% wt.

Preferably, at least one of C1 and C7 is an azeotropic concentration.

As indicated, according to an embodiment of the invention, the feed is a lignocellulosic material feed, which feed comprises lignin. Said lignin does not hydrolyze in the hydrolysis step [B3] and at least a fraction of it does not dissolve in the hydrolyzate. According to that embodiment, in addition to hydrolyzate, said hydrolyzing forms an HCl-comprising lignin stream (<Ig8> in FIG. 1), wherein HCl amount, concentration and purity are W8, C8 and P8, respectively. According to an embodiment, a significant fraction of the HCl of the hydrolysis medium ends up in the lignin stream, so that W8/W3 is greater than 0.2, preferably greater than 0.3, more preferably greater than 0.4 and most preferably greater than 0.5. Such large amounts of HCl are preferably recovered for reuse at a sufficiently high concentration.

In especially preferred embodiments of the present invention, the lignin stream is treated with a fourth organic solvent to extract organic solute wherein said organic solutes are tall oils.

It has been found that the tall oils better dissolve at the highly concentrated acid system and are better recovered before HCl removal from that stream.

Preferably, the method further comprises the step ([D] in FIG. 1) of separating HCl from said HCl-comprising lignin stream to form a third separated HCl stream <3s9> wherein HCl amount, concentration and purity are W9, C9 and P9, respectively, and an HCl-depleted lignin stream <gl>. Preferably said separating comprises distillation and said third separated HCl stream is gaseous. According to an embodiment, at least a portion of said third separated HCl stream is used to form said reagent HCl, e.g. by combining it with at least a portion of said first separated HCl stream and/or a portion of said recovered HCl stream.

In a preferred embodiment azeotropic streams are combined with a lignin stream prior to the distillation.

According to another embodiment, the method further comprises a step ([K] in FIG. 1) of separating HCl from said HCl-depleted lignin stream to form a fourth separated HCl stream <4s10> wherein HCl amount, concentration and purity are W10, C10 and P10, respectively, and an essentially HCl-free lignin stream <fl>. According to an embodiment, chloride concentration in said essentially HCl-free lignin is less than 10,000 ppm, more preferably less than 5000 ppm and most preferably less than 2000 ppm.

According to an embodiment said separating to form said essentially HCl-free lignin comprises contacting said HCl-depleted lignin with a wash stream comprising water or an aqueous stream (e.g. a process stream) low in chloride. According to an embodiment, said contacting is counter-current. According to an embodiment, contacting employs a belt filter. According to an embodiment, said contacting employs a belt filter and a washing contactor operated sequentially. According to an embodiment, the wash stream flows first to the belt filter and then to the washing contactor, while the HCl-depleted lignin is treated first in the washing contactor and then on the belt filter. According to an embodiment, essentially all the HCl-containing aqueous stream formed in the belt filter treatment flows into the washing contactor where it takes up additional HCl to form said fourth recovered HCl stream. According to another embodiment, a portion of the HCl-containing aqueous stream from the belt filter is withdrawn to form a fifth recovered HCl stream, while the rest flows to the washing contactor to form the fourth recovered HCl stream. According to an embodiment, HCl concentration in at least one of said fourth recovered stream, and said fifth recovered stream is lower than azeotropic and said stream is concentrated to azeotropic concentration by distilling water out. According to another embodiment, HCl is extracted out of said fourth recovered stream, out of said fifth recovered stream or both. According to a preferred embodiment, said extraction is conducted by means of the same extractant used for HCl extraction from <dh> or <as1>. According to a related embodiment, said extracting is combined with extracting from those streams. According to one embodiment, at least one of said fourth recovered stream and said fifth recovered stream is mixed with <dh> or <as1> prior to contacting with said organic phase. According to an alternative embodiment, at least one of said fourth recovered stream and said fifth recovered stream is injected into the counter-current extraction of <dh> or <as1>, preferably at a stage where the concentration of the aqueous phases are similar.

According to an embodiment said separating to form said essentially HCl-free lignin comprises distilling HCl and water from said HCl-depleted lignin stream to form a distillate and said essentially HCl-free lignin. According to a preferred embodiment, said distilling is conducted in the presence of a first organic solvent. According to a preferred embodiment, said first organic solvent is volatile and said distillate comprises vapors of water, HCl and said first solvent. According to an embodiment, said distillate is condensed to form an aqueous HCl solution, optionally comprising some of the first solvent. According to a preferred embodiment, said first solvent is of low miscibility in water and in a 20% wt HCl solution. According to said embodiment, on condensing said distillate, two phases are formed, an aqueous HCl solution optionally containing some solvent and a solvent phase, optionally containing some water. According to an embodiment said solvent phase is reused for separating HCl from HCl-depleted lignin.

The present invention further provides an organic phase composition. According to an embodiment, said organic phase is suitable for separating HCl from its aqueous solution, e.g. aqueous solutions also comprising another solute, such as a carbohydrate. The organic phase composition of the present invention comprises: (a) a first component selected from the group consisting of quaternary amines; (b) a second component selected from (b1) the group consisting of category B organic acids, (b2) the group consisting of a mixtures of category B organic acids and category C organic acids at a B/C molar ratio of R_{B/c} and (b3) the group consisting of a mixtures of category A organic acids and category C organic acids at an A/C molar ratio of R_{A/C}; and (c) a third component selected from the group consisting of solvents for said first component and for said second component, wherein (i) all three components are oil-soluble and water-insoluble; (ii) the molar concentration of each of said first component and said second component is greater than 0.6 mol/Kg, preferably greater than 0.8 mol/Kg and more preferably greater than 0.9 mol/Kg; (iii) the molar ratio between said second component and said first component is greater than 0.9 and preferably greater than 1.0; (iv) R_{B/C} and R_{A/C} are greater than 1, preferasbly greater than 1.5 and more preferably greater than 2; (v) category A organic acids are selected from the group consisting of poly-aromatic sulfonic acids, naphthalene sulfonic acids and acids with a pKa in the range within +/−0.5 pKa units of that of naphthalene sulfonic acid; (vi) category B organic acids are selected from the group consisting of mono-aromatic sulfonic acids, benzene sulfonic acids, and acids with a pKa in the range within +/−0.5 pKa units of that of benzene sulfonic acid; and (vii) category C organic acids are selected from the group consisting of phosphoric acid esters and acids with a pKa in the range within +/−0.5 pKa units of that of di-octyl esters of phosphoric acid. According to an embodiment, said category A organic acid is dinonylnaphthalene sulfonic acid. According to an embodiment, said category B organic acid is linear-chain benzylsulfonic acid. According to an embodiment, said category C organic acid is bis-2-ethylhexl phosphoric acid. According to an embodiment, said third component is composed of at least 70% wt. hydrocarbon and said hydrocarbon is an aliphatic hydrocarbon, an aromatic hydrocarbon or a combination thereof.

The pKa of an acid is minus log its dissociation constant. Measuring the pKa of a water soluble acid is straight forward, e.g. by determining the pH or its water solution. The acids of the second component of the present invention are water insoluble. Direct measurement of their pKa is complex and the results may change according to the medium in which it is measured (e.g. its polarity) and the method of measurement. According to a preferred embodiment of the present invention, the pKa of the second component acid is determined by that of a water-soluble analogs, as explained in PCT/IL2009/000392, the relevant teachings of which are incorporated herein by reference. As used herein, an “acid with a pKa in the range within +/−0.5 pKa units of that of another acid” means that the pKa values for the two acids are in that range when measured in the same medium and by the same method.

In a preferred embodiment, the organic phase composition, further comprises HCl and the molar ratio between HCl and said first component is greater than 0.2, preferably greater than 0.5, more preferably greater than 0.8 and most preferably greater than 1.0.

According to various embodiments, said organic phase composition is characterized by the concentration of HCl it assumes (its loading) in equilibration, at a given temperature, with HCl solutions wherein HCl is essentially the only solute or in equilibrium with aqueous solutions of HCl and dextrose. Thus, according to an embodiment, when equilibrated at 45° C. with an aqueous solution containing 35% wt. dextrose and 1% wt. HCl, said organic phase is loaded to at least 0.05 mol/Kg, preferably to at least 0.10 mol/Kg, more preferably to at least 0.15 mol/Kg, and most preferably to at least 0.20 mol/Kg. According to another embodiment, when equilibrated at 90° C. with an aqueous solution containing 2% wt. HCl and no dextrose, said organic phase is loaded to less than 0.30 mol/Kg, preferably to less than 0.25 mol/Kg, more preferably to less than 0.20 mol/Kg and most preferably to less than 0.15 mol/Kg. According to an embodiment, when equilibrated at 45° C. with an aqueous solution containing 35% wt. dextrose and 5% wt. HCl, said organic phase is loaded to between 0.10 mol/Kg and 0.50 mol/Kg, preferably between 0.15 mol/Kg and 0.45 mol/Kg, more preferably between 0.20 mol/Kg and 0.40 mol/Kg. According to anpther embodiment, when equilibrated at 45° C. with an aqueous solution containing 35% wt. dextrose and 10% wt. HCl, said organic phase is loaded to between 0.20 mol/Kg and 0.70 mol/Kg, preferably between 0.25 mol/Kg and 0.65 mol/Kg, more preferably between 0.30 mol/Kg and 0.60 mol/Kg, and most preferably between 0.35 mol/Kg and 0.55 mol/Kg. According to still another embodiment, when equilibrated at 45° C. with an aqueous solution containing 35% wt. dextrose and 15% wt. HCl, said organic phase is loaded to between 0.45 mol/Kg and 0.90 mol/Kg, preferably between 0.50 mol/Kg and 0.85 mol/Kg, more preferably between 0.55 mol/Kg and 0.80 mol/Kg, and most preferably between 0.60 mol/Kg and 0.75 mol/Kg. According to still another embodiment, when equilibrated at 45° C. with an aqueous solution containing 35% wt. dextrose and 20% wt. HCl, said organic phase is loaded to between 0.55 mol/Kg and 1 mol/Kg, preferably between 0.60 mol/Kg and 0.95 mol/Kg, more preferably between 0.65 mol/Kg and 0.90 mol/Kg, and most preferably between 0.70 mol/Kg and 0.85 mol/Kg.

According to an embodiment said organic phase composition provides for both efficient extraction and reversibility as explained above. Preferably, as characteristics to reversible extractants, said organic phase composition is characterized by an essentially linear distribution curve for HCl extraction. More spececifically, said organic phase composition is characterized by an essentially linear distribution curve for HCl extraction from 35% wt. dextrose solution in a range between 1% wt. HCl and 20% wt. HCl.

According to an embodiment, in contacting said organic phase composition also extracts water when contacted with an aqueous solution of HCl. According to an embodiment, said organic phase composition is characterized by a water concentration of between 2.0% and 7.0% when equilibrated at 45° C. with an aqueous solution containing 35% wt. dextrose and 10% wt HCl.

According to another embodiment, after contacting with aqueous HCl solutions and settling, the (loaded) organic phase composition separates well from the formed aqueous solution. According to a specific embodiment, said organic phase composition is characterized by an aqueous/organic phase separation time of less than 5 min as measure after shaking gently 50 times at 50° C. with an aqueous solution containing 35% wt. dextrose and 20% wt HCl.

### EXAMPLES

### Example 1

An extractant was prepared by mixing (i) 1 mol/Kg Aliguat 336™ solution in dodecane with (ii) 1 mol/Kg Lauryl Benzene Sulfonic Acid (LAS) solution in dodecane. 1.5 gr aliquots of the extractant were equilibrated at 23° C. and at 95° C. with 5 gr aqueous HCl solutions. The phases were then separated and analyzed for HCl concentrations (by titration). The results are presented in Table 1. Z herein, and in the following examples, is the molar ratio between the HCl in the extractant and the amine there.

TABLE 1

| 23° C. | | 95° C. | |
|---|---|---|---|
| HCl in | | HCl in | |
| Aqueous (Wt %) | Z | Aqueous (Wt %) | Z |
| 0.5 | 0.02 | 0.5 | 0.01 |
| 3.2 | 0.11 | 3.7 | 0.04 |
| 12.6 | 0.47 | 12.6 | 0.31 |
| 20.6 | 0.75 | 20.6 | 0.57 |
| 7.2 | 0.30 | 7.3 | 0.11 |
| 24.2 | 0.99 | 25.0 | 0.68 |
| 31.3 | 1.43 | 29.8 | 1.27 |

The extractant of this example illustrates an organic phase composition composed of a quaternary amine first component, a category B organic acid second component and an aliphatic hydrocarbon solvent third component, where the molar ratio between the first component and the second one is 1:1 and each of those is at a concentration of 1 mol/Kg. The illustrated molar ratios between the extracted acid and the amine range from 0.02 to 1.43. Comparing equilibria with aqueous solutions of about the same composition, extractant loading are lower at the elevated temperature, indicating improved back-extraction at higher temperatures. In equilibrium with 3.7% HCl aqueous solution at 95° C., the organic phase is loaded to 0.04 mol/Kg.

### Example 2

1.5 gr aliquots of the extractant prepared in Example 1 were equilibrated at 23° C. with 5 gr aqueous HCl solution containing 35% glucose or 65% glucose. The phases were then separated and analyzed for HCl concentrations. The results are summarized in Table 2. Herein and in the following, for aqueous solutions also containing glucose, HCl concentrations are presented on a glucose-free basis, i.e. HCl/(HCl+water).

TABLE 2

| 35% Glucose | | 65% Glucose | |
|---|---|---|---|
| HCl/(HCl + | | HCl/(HCl + | |
| Water) (Wt %) | Z | Water) (Wt %) | Z |
| 2.56 | 0.05 | 2.97 | 0.04 |
| 6.38 | 0.13 | 3.74 | 0.05 |
| 5.52 | 0.13 | 5.03 | 0.13 |

This example illustrates that when the organic phase composition used herein is equilibrated at 23° C. with an aqueous solution containing 35% wt. dextrose and 5.5% wt. HCl, said organic phase is loaded to 0.13 mol/Kg.

### Example 3

An extractant was prepared by mixing Aliguat 336™, Dinonylnaphtalen Sulfonic Acid (DNNS) and a mixtre of hydrophilic hydrocarbon (dodecane and Heptane) at ratios so that the concentrations of the first component and the second component are 0.5 mol/Kg each. 1.5 gr aliquots of the extractant were equilibrated at RT and at 92° C. with 5 gr aqueous HCl solutions. The phases were then separated and analyzed for HCl concentrations (by titration). The results are presented in Table 3.

TABLE 3

| RT | | 92° C. | |
|---|---|---|---|
| HCl in | | HCl in | |
| Aqueous (Wt %) | Z | Aqueous(Wt %) | Z |
| 1.24 | 0.028 | 1.54 | 0.07 |
| 1.81 | 0.023 | 2.6 | 0.09 |
| 4.66 | 0.064 | 6.1 | 0.18 |
| 11.9 | 0.18 | 10.8 | 0.30 |
| 20.4 | 0.57 | | |
| 31.8 | 1.39 | | |

The extractant of this example illustrates an organic phase composition composed of a quaternary amine first component, a category A organic acid second component and an aliphatic hydrocarbon solvent third component, where the molar ratio between the first component and the second one is 1:1. In equilibrium with 2.6% MCI aqueous solution at 92° C., the organic phase is loaded to 0.045 mol/Kg.

### Example 4

An extractant was prepared by mixing Aliguat 336™, DNNS and a mixtre of dodecane, Heptane and 2-Ethyihexanol at ratios so that the concentrations of the first component and of the second component are 0.5 mol/Kg each. Aliquots of the extractant were equilibrated with aqueous HCl solutions as in Example 3. The results are presented in Table 4.

TABLE 4

| RT | | 92° C. | |
|---|---|---|---|
| HCl in | | HCl in | |
| Aqueous(Wt %) | Z | Aqueous(Wt %) | Z |
| 1.14 | 0.03 | 1.14 | 0.08 |
| 2.51 | 0.11 | 2.73 | 0.11 |
| 6.70 | 0.25 | 6.18 | 0.21 |
| 10.7 | 0.44 | 10.4 | 0.37 |
| 19.9 | 1.26 | 19.6 | 1.31 |
| 29.6 | 3.25 | 29.7 | 3.45 |

### Example 5

The extractant of Example 3 and the extractant of Example 4 were tested in extracting HCl from aqueous HCl+Glucose solutions. The procedure was similar to that in the previous examples and the results are presented in Table 5.

TABLE 5

| 2-Ethylhexanol, 35% Glucose | | Dodecane, 20% Glucose | |
|---|---|---|---|
| HCl/(HCl + Water) | | HCl/(HC + Water) | |
| (Wt %) | Z | (Wt %) | Z |
| 1.352 | 0.16 | 4.563 | 0.18 |
| 4.20 | 0.14 | | |
| 8.59 | 0.26 | | |

This example illustrates that when the organic phase compositions used herein are equilibrated at RT with aqueous solutions containing 35% wt. dextrose and 4.2-4.5% wt. HCl, said organic phases are loaded to 0.14-0.18 mol/Kg.

### Example 6

An extractant was prepared by mixing Aliguat 336™, LAS, Di-(2-ethylhexyl)phosphoric acid (DEPHA) and dodecane at ratios so that the concentrations of Aliquate 336, LAS and DEHPA are 1 mol/Kg, 0.75 mol/Kg and 0.25 mol/Kg, respectively. 1.5 gr aliquots of the extractant were equilibrated at 24° C., 50° C. and at 95° C. with 5 gr aqueous HCl solutions. The phases were then separated and analyzed for HCl concentrations. The results are presented in Table 6

TABLE 6

| 24° C. | | 50° C. | | 95° C. | |
|---|---|---|---|---|---|
| HCl in | | HCl in | | HCI in | |
| Aqueous (Wt %) | Z | Aqueous (Wt %) | Z | Aqueous (Wt %) | Z |
| 0.5 | 0.13 | 0.37 | 0.08 | 0.2 | 0.07 |
| 2.3 | 0.24 | 0.66 | 0.17 | 0.5 | 0.06 |
| 6.2 | 0.38 | 1.76 | 0.23 | 2.3 | 0.12 |
| 8.8 | 0.50 | 2.67 | 0.31 | 6.2 | 0.23 |
| 15.8 | 0.73 | 4.88 | 0.34 | 8.8 | 0.32 |
| 22.7 | 1.01 | | | 15.8 | 0.61 |
| 32.0 | 1.56 | | | 22.7 | 0.97 |
| | | | | 32.0 | 1.49 |
| | | | | 22.7 | 0.89 |

The extractant of this example illustrates an organic phase composition composed of a quaternary amine first component, a mixture of category B organic acid and a category C organic acid second component and an aliphatic hydrocarbon solvent third component. The molar ratio between the first component and the second one is 1:1, each of those is at a concentration of 1 mol/kg and the ratio between the category B organic acid and the category C organic acid is 3:1. The extraction results demonstrates the reduced extraction at 95° C. compared with that at 24° C., i.e. improved back-extraction on temperature elevation.

### Example 7

The extractant formed in Example 6 was used for the extraction of HCl from aqueous HCl solutions containing varied concentrations of glucose. Extraction temperature was 45° C. Table 7 and FIG. 2 present the results of HCl distribution between the phases. Water co-extraction into the organic phase was checked for some of the equilibrations with 20% glucose and 35% glucose aqueous phases. The results are presented in Table 8.

TABLE 7

| 20% Glucose | | 35% Glucose | | 50% Glucose | | 65% Glucose | |
|---|---|---|---|---|---|---|---|
| HCl/(HCl + | | HCl/(HCl + | | HCl/(HCl + | | HCl/(HCl + | |
| Water) | | Water) | | Water) | | Water) | |
| (Wt %) | Z | (Wt %) | Z | (Wt %) | Z | (Wt %) | Z |
| 0.61 | 0.15 | 0.69 | 0.15 | 0.79 | 0.14 | 0.475 | 0.08 |
| 2.08 | 0.17 | 1.08 | 0.19 | 1.4 | 0.12 | 3.24 | 0.16 |
| 7.45 | 0.34 | 1.92 | 0.20 | 6.5 | 0.26 | 6.02 | 0.24 |
| 10.92 | 0.46 | 5.01 | 0.32 | 8.1 | 0.35 | 11.31 | 0.40 |
| 14.68 | 0.66 | 7.44 | 0.37 | 11.7 | 0.44 | 13.79 | 0.47 |
| 16.50 | 0.70 | 11.91 | 0.51 | 15.8 | 0.57 | 17.25 | 0.59 |
| 21.39 | 0.93 | 14.64 | 0.60 | 18.2 | 0.67 | 22.36 | 0.78 |
| | | 22.09 | 0.89 | 23.6 | 0.84 | | |
| | | 22.58 | 0.97 | | | | |

TABLE 8

| 20% Glucose | | 35% Glucose | |
|---|---|---|---|
| H₂0 (Wt %) | Z | H₂0 (Wt %) | Z |
| 4.30 | 0.17 | 3.41 | 0.15 |
| 5.87 | 0.93 | 3.70 | 0.19 |
| | | 5.25 | 0.89 |

FIG. 2 illustrates an essentially linear distribution curve for the extraction of HCl from 35% glucose solution at 45° C.

When equilibrated at 45° C. with an aqueous solution containing 35% wt. dextrose and 0.69-22.1% wt HCl, the water content of the organic phase was in the range between 3.4 and 5.2%.

### Example 8

The extractant formed in Example 6 and an aqueous solution containing 35% wt. dextrose and 20% wt HCl were introduced into a test tube and the temperature was adjusted to 45° C. The test tube was shaken gently 50 times and then allowed to settle. Two clear phases were observed within less than a minute and phase separation was completed within about 2 minutes.

### Example 9

An extractant was prepared by mixing Aliguat 336™, DEPHA and LAS in decane or dodecane to reach concentrations of 1.0, 0.15 and 0.85 mol/Kg, respectively. 1.5 gr aliquots of the extractant were equilibrated at RT and at 95° C. with 5 gr aqueous HCl solution containing 35% glucose and containingno glucose, respectively. The phases were then separated and analyzed for HCl concentrations. The results are summarized in Table 9.

TABLE 9

| decane, 35% Glucose, RT | | Dodecane, 95° C. | |
|---|---|---|---|
| HCl/(HCl + | | HCl in | |
| Water) (Wt %) | Z | Aqueous (Wt %) | Z |
| 0.61 | 0.05 | 0.66 | 0.12 |
| 1.14 | 0.14 | 1.53 | 0.19 |
| 4.82 | 0.23 | 5.64 | 0.22 |
| 8.66 | 0.32 | 9.47 | 0.33 |
| 12.25 | 0.48 | 16.18 | 0.59 |
| 17.06 | 0.62 | 22.22 | 0.87 |
| 22.79 | 0.85 | | |

The extractant of this example illustrates an organic phase composition composed of the same components as those of Examples 6 and 7, but the ratio between the category B organic acid and the category C organic acid is 5.7:1.

### Example 10

An extractant was prepared by mixing Aliguat 336™, DEPHA and LAS in decane to reach concentrations of 1.0, 0.40 and 0.60 mol/Kg, respectively. 1.5 gr aliquots of the extractant were equilibrated at RT and at 95° C. with 5 gr aqueous HCl solution containing 35% glucose and containing no glucose, respectively. The phases were then separated and analyzed for HCl concentrations. The results are summarized in Table 10.

TABLE 10

| 35% Glucose, RT | | 95° C. | |
|---|---|---|---|
| HCl/(HCl + Water) | | HCl in | |
| (Wt %) | Z | Aqueous (Wt %) | Z |
| 0.98 | 0.21 | 0.6 | 0.19 |
| 4.34 | 0.30 | 2.8 | 0.31 |
| 9.26 | 0.50 | 6.1 | 0.40 |
| 17.40 | 0.75 | 19.2 | 0.52 |
| 32.06 | 1.42 | 32.0 | 1.87 |

### Example 11

An extractant was prepared by mixing Aliguat 336™, DEPHA and LAS in decane to reach concentrations of 0.68, 0.21 and 0.59 mol/Kg, respectively. The extractants were loaded with HCl to reach Z of 1. N₂ was bubbled at a constant rate of about 10 ml/min through the extractants at selected temperatures for selected time periods after which the HCl concentration in the extractant was analyzed by titration. The changes in HCl concentrations were translated into HCl partial vapor pressures. The results are presented in Table 11.

TABLE 11
0.68 mo1/Kg Aliguat 336 ™ 0.21 mol/Kg
DEPHA, and 0.59 mo1/Kg LAS in Decane

| Time (Min.) | Temp. (° C.) | N2 ml/min | HCl Z | HCl mmAg |
|---|---|---|---|---|
| 15 | 90 | 17 | 0.71 | 12.1 |
| 10 | | | 0.64 | 6.0 |
| 25 | | | 0.45 | 6.0 |
| 35 | | | 0.32 | 2.3 |
| 5 | 103 | 10 | 0.78 | 58 |
| 7 | | | 0.68 | 25 |
| 8.5 | | | 0.64 | 8.6 |
| 35 | | | 0.56 | 3.5 |
| 75 | | | 0.34 | 3.5 |
| 1 | 121 | 10 | 0.61 | 236 |
| 4 | | | 0.52 | 28 |
| 14 | | | 0.34 | 16 |
| 32 | | | 0.16 | 5.8 |
| 45 | | | 0.12 | 0.60 |
| 45 | | | 0.09 | 0.28 |
| 1 | 128 | 10 | 0.75 | 294 |
| 4 | | | 0.60 | 65 |
| 7 | | | 0.40 | 45 |
| 14 | | | 0.23 | 17 |
| 35 | | | 0.10 | 5 |
| 65 | | | 0.08 | 0.24 |

The results in Table 11 demonstrate efficient distillation of a large fraction of the extracted HCl.

It will be evident to those skilled in the art that the invention is not limited to the details of the foregoing illustrative embodiments and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. An organic phase composition comprising:
a. a first component selected from the group consisting of quaternary amines;
b. a second component selected from:
b1. the group consisting of category B organic acids;
b2. the group consisting of a mixtures of category B organic acids and category C organic acids at a B/C molar ratio of R_{B/C}; and
b3. the group consisting of a mixtures of category A organic acids and category C organic acids at an A/C molar ratio of R_{A/C},
c. a third component selected from the group consisting of solvents for said first component and for said second component, wherein
(i) all three components are oil-soluble and water-insoluble;
(ii) the molar concentration of each of said first component and said second component is greater than 0.6 mol/Kg;
(iii) the molar ratio between said second component and said first component is greater than 0.9;
(iv) R_{B/C} and R_{A/C} are greater than 2;
(v) category A organic acids are selected from the group consisting of poly-aromatic sulfonic acids, naphthalene sulfonic acids and acids with a pKa in the range within +/−0.5 pKa units of that of naphthalene sulfonic acid;
(vi) category B organic acids are selected from the group consisting of mono-aromatic sulfonic acids, benzene sulfonic acids, and acids with a pKa in the range within +/−0.5 pKa units of that of benzene sulfonic acid; and
(vii) category C organic acids are selected from the group consisting of phosphoric acid esters and acids with a pKa in the range within +/−0.5 pKa units of that of di-octyl esters of phosphoric acid.

2. (canceled)

3. The organic phase composition according to claim 1, further comprising HCl wherein the molar ratio between HCl and said first component is greater than 1.0.

4. (canceled)

5. The organic phase composition according to claim 1, characterized in that when equilibrated at 45° C. with an aqueous solution containing 35% wt. dextrose and 1% wt. HCl, said organic phase is loaded to at least 0.05 mol/Kg; in that when equilibrated at 90° C. with an aqueous solution containing 2% wt. HCl, said organic phase is loaded to less than 0.30 mol/Kg; and in that when equilibrated at 45° C. with an aqueous solution containing 35% wt. dextrose and 5% wt. HCl, said organic phase is loaded to between 0.10 mol/Kg and 0.50 mol/Kg; in that when equilibrated at 45° C. with an aqueous solution containing 35% wt. dextrose and 10% wt. HCl, said organic phase is loaded to between 0.20 mol/Kg and 0.70 mol/Kg; in that when equilibrated at 45° C. with an aqueous solution containing 35% wt. dextrose and 15% wt. HCl, said organic phase is loaded to between 0.45 mol/Kg and 0.90 mol/Kg; and in that when equilibrated at 45° C. with an aqueous solution containing 35% wt. dextrose and 20% wt. HCl, said organic phase is loaded to between 0.55 mol/Kg and 1 mol/Kg.

6. (canceled)

7. The organic phase composition according to claim 1, characterized by an essentially linear distribution curve for HCl extraction from 35% wt. dextrose solution in a range between 1% wt. HCl and 20% wt. HCl.

8. The organic phase composition according to claim 1, characterized by a water concentration of between 2.0% and 7.0% when equilibrated at 45° C. with an aqueous solution containing 35% wt. dextrose and 10% wt HCl.

9. The organic phase composition according to claim 1, characterized by an aqueous/organic phase separation time of less than 5 min as measure after shaking gently 50 times at 50° C. with an aqueous solution containing 35% wt. dextrose and 20% wt HCl.

10. A method for the recovery of HCl comprising
a. bringing an aqueous process stream comprising HCl and a solute, wherein HCl amount, concentration and purity are W1, C1 and P1, respectively, into contact at a temperature T1 with an organic phase according to claim 1, whereupon HCl selectively transfers to said organic phase to form an HCl-carrying extract; and
b. recovering, at a temperature T2, HCl from said HCl-carrying solvent to form a recovered HCl stream wherein HCl amount, concentration and purity are W2, C2 and P2, respectively, and a regenerated organic phase, wherein said recovering comprises at least one of:
b1. bringing said HCl-carrying extract into contact with an aqueous back-extracting stream, whereupon HCl transfers to said aqueous stream, and
b2. distilling HCl from said HCl-carrying extract
wherein
(i) W2/W1 is greater than 0.5
(ii) C2/C1 is greater than 0.7;
(iii) P2/P1 is greater than 20; and
(iv) T1 and T2 are less than 130° C.

11. The method according to claim 10, wherein said recovering comprises both bringing in contact and distilling and wherein said distilling precedes said bringing in contact.

12. The method according to claim 10, wherein T2 is greater than T1 by at least 20° C.

13. The method according to claim 10, wherein said solute is a carbohydrate, said carbohydrate concentration is greater than 15% wt. and said process stream is formed in a process of hydrolyzing a polysaccharide in a polysaccharide-containing material.

14. The method according to claim 10, further comprising a step of using said recovered HCl stream for the hydrolysis of a polysaccharide.

15. A method for the production of carbohydrate comprising
a) providing a feed comprising a polysaccharide;
b) hydrolyzing said polysaccharide in an HCl-comprising hydrolysis medium, wherein HCl amount, concentration and purity are W3, C3 and P3, respectively, to form a hydrolyzate comprising carbohydrate and HCl, wherein HCl amount, concentration and purity are W4, C4 and P4, respectively;
c) separating a portion of the HCl from said hydrolyzate to form a first separated HCl stream wherein HCl amount, concentration and purity are W5, C5 and P5, respectively, and an HCl-depleted hydrolyzate, wherein HCl amount, concentration and purity are W1, C1 and P1, respectively;
d) bringing said HCl-depleted hydrolyzate into contact at a temperature T1 with an organic phase according to claim 1, whereupon HCl selectively transfers to said organic phase to form an HCl-carrying extract and an essentially HCl-free hydrolyzate;
e) recovering, at a temperature of T2, HCl from said HCl-carrying solvent to form a recovered HCl stream wherein HCl amount, concentration and purity are W2, C2 and P2, respectively, and a regenerated organic phase, wherein said recovering comprises at least one of
e1. bringing said HCl-carrying extract into contact at a temperature T2 with an aqueous back-extracting stream, whereupon HCl transfers to said aqueous stream, and
e2. distilling HCl from said HCl-carrying extract;
f) combining at least a portion of said first separated HCl stream and a portion of said recovered HCl stream to form a reagent HCl stream, wherein HCl amount, concentration and purity are W6, C6 and P6, respectively; and
g) combining said reagent HCl stream with a lignocellulosic material to form said hydrolysis medium;
wherein
i. said first HCl stream is essentially carbohydrate free;
ii. W2/W1 is greater than 0.5, W1/W3 and W1/W4 are each less than 0.4, W5/W4 is greater than 0.1, and W6/W4 is greater than 1;
iii. C2/C1 is greater than 0.7;
iv. C3, C4, C5 and C6 are each greater than 30% wt;
v. P2/P1 is greater than 20, and P3/P1 and P4/P1 are each greater than 50;
vi. P5 and P6 are each greater than 80%; and
vii. T1 and T2 are each less than 130° C.

16. The method according to claim 15, wherein said recovering comprises both bringing in contact and distilling and wherein said distilling precedes said bringing in contact.

17. The method according to claim 15, wherein T2 is greater than T1 by at least 20° C.

18. (canceled)

19. The method according to claim 15, wherein C3 is greater than 36% wt, and C4, C5 and C6 are greater than 39% wt.

20-21. (canceled)

22. The method according to claim 15, wherein said feed comprises a lignocellulosic material, wherein said hydrolyzing further forms an HCl-comprising lignin stream, wherein HCl amount, concentration and purity are W8, C8 and P8, respectively, wherein W8/W3 is greater than 0.2, C8 is greater than 35% and P8/P1 is greater than 20; and wherein the method further comprises the steps of:
h) separating HCl from said HCl-comprising lignin stream to form a third separated HCl stream wherein HCl amount, concentration and purity are W9, C9 and P9, respectively, and an HCl-depleted lignin stream; and
i) combining at least a portion of said third separated HCl stream with at least a portion of said first separated HCl stream and a portion of said recovered HCl stream to form said reagent HCl stream, wherein C9 is greater than 30% wt, P9 is greater than 80% wt and W9/W8 is greater than 0.1.

23-24. (canceled)

25. The method according to claim 22, further comprising a step of;
j) separating HCl from said HCl-depleted lignin stream to form a fourth separated HCl stream wherein HCl amount, concentration and purity are W10, C10 and P10, respectively, and an essentially HCl-free lignin stream and wherein W10/W8 is greater than 0.1, C10 is greater than 10% wt and P10 is greater than 50% wt.; and wherein said separating HCl from said HCl-depleted lignin stream comprises distilling HCl in the presence of a first organic solvent.

26-28. (canceled)

29. A method for the production of a carbohydrate comprising;
a) providing a lignocellulosic material feed comprising a polysaccharide;
b) hydrolyzing said polysaccharide in an HCl-comprising hydrolysis medium, wherein HCl amount, concentration and purity are W3, C3 and P3, respectively, to form a hydrolyzate comprising carbohydrate and HCl, wherein HCl amount, concentration and purity are W4, C4 and P4, respectively and an HCl-comprising lignin stream, wherein HCl amount, concentration and purity are W8, C8 and P8, respectively;
c) separating a portion of the HCl from said hydrolyzate to form a first separated HCl stream wherein HCl amount, concentration and purity are W5, C5 and P5, respectively, and an HCl-depleted hydrolyzate, wherein HCl amount, concentration and purity are W1, C1 and P1, respectively;
d) separating HCl from said HCl-comprising lignin stream to form a third separated HCl stream wherein HCl amount, concentration and purity are W9, C9 and P9, respectively, and an HCl-depleted lignin stream; and
e) bringing said HCl-depleted hydrolyzate into contact at a temperature T1 with an organic phase, according to claim 1,
whereupon HCl selectively transfers to said organic phase to form an HCl-carrying extract and an essentially HCl-free hydrolyzate;
f) recovering, at a temperature of T2, HCl from said HCl-carrying extract to form a recovered HCl stream wherein HCl amount, concentration and purity are W2, C2 and P2, respectively, and a regenerated organic phase, wherein said recovering comprises at least one of
f1. bringing said HCl-carrying extract into contact with an aqueous back-extracting stream, whereupon HCl transfers to said aqueous stream and
f2. distilling HCl from said HCl-carrying extract;
g) combining at least a portion of said first separated HCl stream, a portion of said recovered HCl stream and a portion of said third separated HCl stream to form a reagent HCl stream, wherein HCl amount, concentration and purity are W6, C6 and P6, respectively; and
h) combining said reagent HCl stream with a lignocellulosic material to form said hydrolysis medium; wherein
(i) said first HCl stream is essentially carbohydrate free;
(ii) W2/W1 is greater than 0.5, W1/W3 and W1/W4 are each less than 0.4, W5/W4 is greater than 0.1, W6/W4 is greater than 1, W8/W3 is greater than 0.2 and W9/W8 is greater than 0.1;
(iii) C2/C1 is greater than 0.7;
(iv) C3, C4, C5, C6, C8 and C9 are each greater than 30% wt;
(v) P2/P1 and P8/P1 are each greater than 20 and, P3/P1 and P4/P1 are each greater than 50;
(vi) P5, P6 and P9 are each greater than 80% and
(vii) T1 and T2 are each less than 130° C.;
(viii) each of said contacts comprises at least 3 counter-current stages.

30. (canceled)

31. The method according to claim 29, wherein T2 is greater than T1 by at least 20° C.

32. The method according to claim 29 further comprising the steps of:
i) separating HCl from said HCl-depleted lignin stream to form a fourth separated HCl stream wherein HCl amount, concentration and purity are W10, C10 and P10, respectively, and an essentially HCl-free lignin stream; and
i) combining at least a portion of said fourth separated HCl stream with at least a portion of said first separated HCl stream, a portion of said recovered HCl stream and a portion of said third separated HCl stream to form said reagent HCl stream, wherein W10/W8 is greater than 0.1, C10 is greater than 10% wt and P10 is greater than 50% wt., wherein said separating HCl from said HCl-depleted lignin stream comprises distilling HCl in the presence of a first organic solvent.

33-34. (canceled)

35. The method according to any of claim 29, wherein C3, C4, C5, C6 and C9 are each greater than 39% wt.

36-40. (canceled)

41. The method according to claim 29, wherein said hydrolyzate further comprises an organic solute, and the method further comprises the step of:
i) bringing said hydrolyzate into contact at a temperature T3 with a third organic solvent, whereupon said organic solute selectively transfers to said third organic solvent to form an organic solute-depleted hydrolyzate and a first organic solute-carrying solvent; and optionally:
i) recovering said third solvent and organic solute from said first extractives-carrying solvent to form a separated organic solute and a regenerated third solvent.

42-45. (canceled)
